(19) United States
(12) Patent Application Publication
(10) Pub. No.: US 2024/0197543 A1
(43) Pub. Date: Jun. 20, 2024

(54) MEDICAL SYSTEMS, DEVICES, AND RELATED METHODS

(71) Applicant: BOSTON SCIENTIFIC MEDICAL DEVICE LIMITED, Galway (IE)

(72) Inventors: Sharath Kumar G, Kanakapura (IN); Deepak Kumar SHARMA, Muzaffarnagar (IN)

(73) Assignee: BOSTON SCIENTIFIC MEDICAL DEVICE LIMITED, Galway (IE)

(21) Appl. No.: 18/541,175

(22) Filed: Dec. 15, 2023

Related U.S. Application Data
(60) Provisional application No. 63/387,758, filed on Dec. 16, 2022.

(51) Int. Cl. A61F 13/26; A61B 1/00; A61B 1/273

(52) U.S. Cl. CPC A61F 13/266; A61B 1/00066; A61B 1/00087; A61B 1/00101; A61B 1/2736; A61F 2013/2014

(57) ABSTRACT

A medical device comprises a handle, wherein the handle includes a primary actuator and a secondary actuator, an end cap configured to be coupled to a distal end of another medical device, wherein the end cap includes a stationary portion and a movable portion, a patch, wherein the patch is coupled to the movable portion of the end cap, one or more control elements coupling the primary actuator to the movable portion of the end cap, and an actuation element, wherein the actuation element extends distally from the secondary actuator and the actuation element is configured to be coupled to the patch, wherein movement of the primary actuator moves the movable portion of the end cap relative to the stationary portion of the end cap, and wherein movement of the secondary actuator at least partially deploys the patch from the movable portion of the end cap.

### CROSS-REFERENCE TO RELATED APPLICATIONS

This application claims the benefit of priority under 35 U.S.C. § 119 from U.S. Provisional Application No. 63/387,758, filed Dec. 16, 2022, which is incorporated by reference herein in its entirety.

### TECHNICAL FIELD

The disclosure relates generally to systems, devices, and methods for delivering patches. More specifically, aspects of the disclosure pertain to systems, devices, and/or methods for delivering patches, for example, for hemostasis, via medical devices, such as endoscopes.

### BACKGROUND

Bleeding ulcers, for example, in a subject's gastrointestinal (GI) tract, are often difficult to manage and/or provide hemostasis. For example, common treatments for bleeding ulcers include injection therapies, thermal therapies, mechanical therapies, and hemostatic powders. Such therapies are often expensive and/or time-consuming. Furthermore, such therapies may not be able to treat a larger surface area, for example, a larger ulcer in the GI tract. Additionally, a common treatment for chronic ulcers is a gastric bypass. Such procedures may be more difficult, more time-consuming, more costly, and/or less effective/accurate than a minimally-invasive procedure to position a patch on one or more ulcers. Therefore, a need exists for systems, devices, and/or methods for positioning and/or deploying one or more hemostatic patches with one or more portions of a subject.

### SUMMARY

This disclosure includes medical systems and devices comprising a biocompatible patch and methods of use thereof, e.g., methods of delivering a patch to a target site of a subject, for example, to help heal an ulcer and/or to perform hemostasis. Each of the aspects disclosed herein may include one or more of the features described in connection with any of the other disclosed aspects.

In one or more examples, a medical device may comprise a handle, wherein the handle includes a primary actuator and a secondary actuator, an end cap configured to be coupled to a distal end of another medical device, wherein the end cap includes a stationary portion and a movable portion, a patch, wherein the patch is coupled to the movable portion of the end cap, one or more control elements coupling the primary actuator to the movable portion of the end cap, and an actuation element, wherein the actuation element extends distally from the secondary actuator and the actuation element is configured to be coupled to the patch, wherein movement of the primary actuator moves the movable portion of the end cap relative to the stationary portion of the end cap, and wherein movement of the secondary actuator at least partially deploys the patch from the movable portion of the end cap.

According to another example, the handle may include a handle body, and wherein both the primary actuator and the secondary actuator are movable relative to the handle body to control the movement of the one or more control elements and the actuation element. The handle body may include a track and the primary actuator is movably coupled to track, and the primary actuator may be configured to translate along the track in a first direction and an opposite second direction. A translation of the primary actuator in the first direction may be configured to translate the movable portion of the end cap distally relative to the stationary portion, and a translation of the primary actuator in the second direction may be configured to translate the movable portion of the end cap proximally relative to the stationary portion.

According to another example, the patch may comprise a first edge and an opposite second edge, wherein each of the first edge and the second edge includes a channel extending along at least a portion of the first edge or the second edge. The actuation element may include a proximal portion and a distal portion, and the distal portion may be configured to be coupled to the patch. The distal portion may include a first strand and a second strand, and the channel of the first edge of the patch may be configured to receive the first strand and the channel of the second edge of the patch may be configured to receive the second strand. The handle body may include a track and the secondary actuator may be movably coupled to track, and the secondary actuator may be configured to translate along the track in a first direction, and the translation of the secondary actuator in the first direction may release the actuation element from the patch and at least partially deploy the patch. The translation of the secondary actuator in the first direction may pull the actuation element in a proximal direction away from the patch, and may retract the first strand from the channel of the first edge of the patch and the second strand from the channel of the second edge of the patch.

According to another example, each of the first edge and the second edge of the patch may include one or more anchors, wherein each of the one or more anchors is configured to anchor the patch onto a treatment site. Each of the one or more anchors may comprise a hook.

According to another example, the patch may comprise one or more wires, wherein each of the one or more wires extend throughout a length of the patch, and wherein the one or more wires bias the patch into a shape. The one or more wires may be biased to a straight shape or to a circular shape.

According to another example, the stationary portion of the end cap may include one or more through-holes, and the one or more control elements and the actuation element may extend through the corresponding one of the one or more first through-holes of the stationary portion of the end cap.

According to another example, the medical device may further comprise a sheath configured to at least partially cover the patch and the end cap, and the sheath may be retractable and/or the movable portion may be extendable to expose the patch and at least the movable portion of the end cap.

In one or more examples, a medical device may comprise a handle, wherein the handle includes a primary actuator and a secondary actuator, an end cap configured to be coupled to a distal end of another medical device, wherein the end cap includes a stationary portion and a movable portion, a patch, wherein the patch is coupled to the movable portion of the end cap, one or more control elements coupling the primary actuator to the movable portion of the end cap, and an actuation element extending distally from the secondary actuator, wherein the actuation element includes a distal portion including a first strand and a second strand, and wherein the patch includes a first channel configured to receive the first strand and a second channel configured to receive the second strand, wherein a movement of the secondary actuator is configured to retract the first strand from the first channel and the second strand from the second channel and at least partially deploy the patch from the movable portion of the end cap.

According to an example, movement of the primary actuator may move the movable portion of the end cap relative to the stationary portion of the end cap. The medical system may further comprise a locking mechanism, wherein the locking mechanism comprises a rotatable knob, and wherein a rotational movement of the rotatable knob secures the primary actuator in a locked position.

In one or more examples, a medical system may comprise a scope, wherein the scope includes a distal portion, a medical device, wherein the medical device includes a handle, wherein the handle includes a primary actuator and a secondary actuator, an end cap coupled to the distal portion of the scope, wherein the end cap includes a stationary portion and a movable portion, a patch coupled to the movable portion of the end cap, wherein the patch includes a first channel and a second channel, one or more control elements coupling the primary actuator to the movable portion of the end cap, and an actuation element extending distally from the secondary actuator, wherein the actuation element includes a first strand and a second strand, wherein the first strand is configured to be received by the first channel and the second strand is configured to be received by the second channel, wherein movement of the primary actuator moves the movable portion of the end cap relative to the stationary portion of the end cap, and wherein movement of the secondary actuator at least partially deploys the patch from the movable portion of the end cap. The handle may include a handle body, wherein the handle body includes a track, wherein both the primary actuator and the secondary actuator are movably coupled to the track, wherein both the primary actuator and the secondary actuator are movable relative to the handle body, along the track, to control the movement of the one or more control elements and the actuation element, and wherein the movement of the secondary actuator in a first direction releases the actuation element from the patch and at least partially deploys the patch.

Any of the examples described herein may have any of these features in any combination.

It may be understood that both the foregoing general description and the following detailed description are exemplary and explanatory only and are not restrictive of the invention, as claimed. As used herein, the terms “comprises,” “comprising,” “having,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” (arrow D) refers to a direction away from an operator/toward a treatment site, and the term “proximal” (arrow P) refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts a perspective view of an exemplary medical device.

FIG. 2 depicts a cross-sectional view of a portion of the exemplary medical device of FIG. 1.

FIGS. 3A and 3B depict perspective views of the exemplary medical device of FIG. 1.

FIG. 4A depicts a perspective view of a portion of the distal end of the exemplary medical device of FIG. 1, and FIG. 4B depicts a different perspective view of a portion of the distal end of the exemplary medical device of FIG. 1.

FIG. 5 illustrates a flow chart of operating the exemplary medical device of FIG. 1.

### DETAILED DESCRIPTION

Reference is now made in detail to examples of this disclosure, aspects of which are illustrated in the accompanying drawings. Wherever possible, the same reference numbers will be used throughout the drawings to refer to the same or like parts.

Embodiments of this disclosure seek to improve a user's ability to position and/or deploy a patch within a subject's body during a medical procedure, help reduce the need to remove a scope, e.g., an endoscope, and reintroduce another medical device into the subject's body, help perform hemostasis within the subject, and reduce overall procedure time, among other aspects.

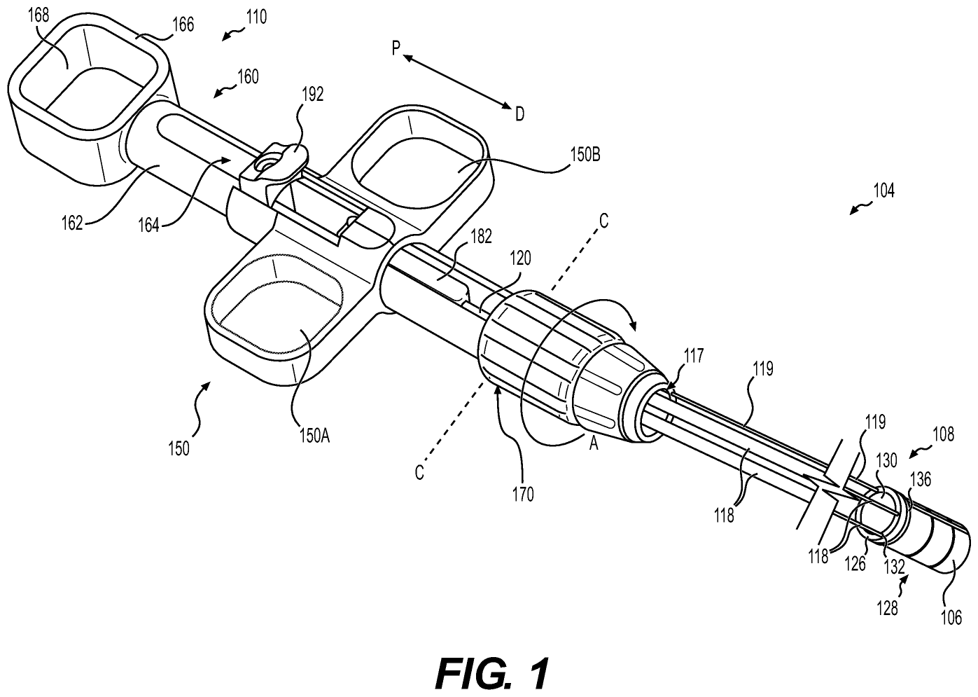

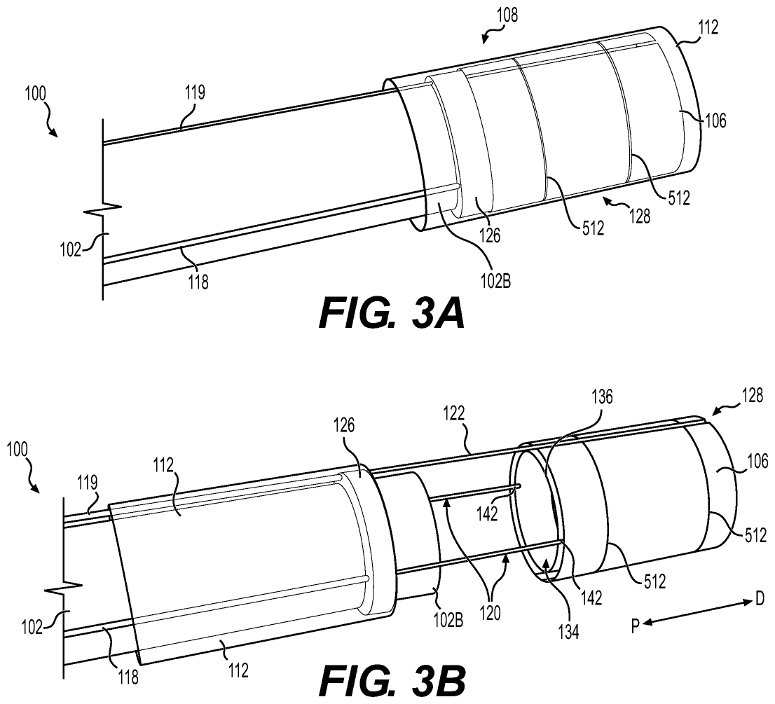

FIG. 1 illustrates a medical device 104. As shown in FIGS. 3A and 3B, one or more portions of medical device 104 may be coupled to another or second medical device, for example, an endoscope 102, to form a medical system 100. As discussed in detail herein, medical device 104 may include or otherwise be coupled to a patch delivery system, for example, to position and/or deliver a patch 106 to one or more portions of tissue within a subject, which may help perform hemostasis within the subject. Additionally, medical system 100 may include a protective element or a sheath 112 (shown in FIGS. 3A and 3B). Sheath 112 may be at least partially transparent, and may be movably positioned radially outside of patch 106, for example, during the delivery of one or more portions of medical system 100 to the treatment site. Sheath 112 may be proximally retracted to expose patch 106, and one or more portions of medical device 104 may be actuated to position and/or deploy patch 106. The mechanism(s) by which sheath 112 may be retracted is not particularly limited, and may include, for example, a pull string or wire. In some other aspects, a proximal end of sheath 112 may be manually manipulated should the proximal end of sheath 112 be accessible to the user for manual retraction.

Medical device 104 may be coupled to one or more portions of endoscope 102, for example, in order to deliver one or more portions of medical device 104 to a treatment site. For example, medical device 104 may include an end cap 108. End cap 108 may be coupled a distal portion 102B (i.e., a distal end) of endoscope 102, such that end cap 108 and distal portion 102B of endoscope 102 may be delivered to the treatment site. Furthermore, it is noted that one or more portions, or an entirety, of end cap 108 may be transparent, which may help the user to visualize the treatment site and/or patch 106, for example, with one or more visualization device(s) and/or illumination device(s) on endoscope 102 (e.g., on a distal end face of endoscope 102). In some aspects, cap 108 may be formed of an acrylic material.

Medical device 104 may include a handle 110, and manipulation of one or more portions of handle 110 may help to maneuver, position and/or reposition, release or deploy, and/or deliver patch 106, for example, to position patch 106 over tissue at the treatment site. As discussed below, features of handle 110 may be coupled to end cap 108 via one or more control element(s) 120 and one or more actuation elements(s) 122.

Endoscope 102 may include a generally cylindrical tubular shape, and may include a proximal portion (not shown) and distal portion 102B. Although not shown, the proximal portion may include or otherwise be coupled to a handle, for example, including one or more ports, controls, levers, electrical or communication connections, etc. Additionally, endoscope 102 may include one or more internal lumens or working channels, for example, extending longitudinally through endoscope 102. In these aspects, the internal lumens or working channels may extend through the proximal portion and distal portion 102B, for example, terminating distally at one or more distal openings (i.e., in a distal most end of endoscope 102). As shown in FIGS. 3A-3B, when medical device 104, including patch 106, is coupled to endoscope 102 (i.e., during the delivery and positioning), patch 106 and a distal portion of medical device 104 may be coupled to distal portion 102B of endoscope 102, for example, coupled to and/or extending distally beyond the distal opening(s) of the internal lumen(s) or otherwise extend distally beyond distal portion 102B.

Endoscope 102 (i.e., distal portion 102B of endoscope 102) is not particularly limited, and may include a diameter of approximately 9 mm to approximately 15 mm, for example, approximately 10.5 mm to approximately 12 mm. One or more portions of end cap 108 may include a size and/or shape configured to be coupled to (e.g., radially surround) distal portion 102B of endoscope 102. As mentioned, endoscope 102 may include one or more internal lumens, for example, a working channel with a diameter of approximately 2 mm to approximately 4 mm, for example, approximately 2.8 mm. Additionally, endoscope 102, for example, a distal end face (not shown) of endoscope 102, may include one or more illumination device(s) (e.g., one or more LEDs, optical fibers, and/or other illuminators) and/or one or more visualization device(s) (e.g., one or more cameras, one or more image sensors, endoscopic viewing elements, optical assemblies including one or more image sensors and one or more lenses, etc.).

Although not shown, one or more portions of endoscope 102 (i.e., distal portion 102B) may be deflectable, for example, via one or more knobs or other controls on a proximal handle. In these aspects, distal portion 102B of endoscope 102 may be maneuvered while being delivered to the treatment site and/or positioned relative to the treatment site, for example, in a retroflex position, which may be used when the treatment site is in the subject's esophagus, stomach, duodenum, colon, or other portion of the GI tract.

Although the treatment site is discussed as being in the subject's GI tract, this disclosure is not so limited, as the treatment site may be any internal lumen or other tissue within the subject. Additionally, although endoscopes are referenced herein, it will be appreciated that the disclosure encompasses any medical devices having a working channel extending from a proximal end to a distal end, such as ureteroscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, or catheters.

Additionally, in some aspects, medical system 100 may include sheath 112, for example, an outer sheath (FIGS. 3A-3B). Sheath 112 may be movable and may surround one or more portions of endoscope 102 and medical device 104, for example, during the delivery of endoscope 102 (distal portion 102B) and end cap 108 to the treatment site. Sheath 112 may help to cover patch 106 during the delivery of endoscope 102 and end cap 108 to the treatment site. For example, as shown in FIGS. 3A-3B, sheath 112 may be proximally retracted once endoscope 102 and end cap 108 are positioned at the treatment site, exposing patch 106.

Patch 106 may be a biodegradable and/or biocompatible patch of any suitable shape and any suitable dimension, for example, based on the nature of the target tissue site. Patch 106 may be flexible and may have any shape such as, for example, approximately square, approximately rectangular, square or rectangular with rounded edges, ovate, circular, among other possible shapes. In some examples, the thickness of the patch may be on the order of millimeters, for example, ranging from approximately 0.1 mm to approximately 5.0 mm or, more specifically, from approximately 0.7 mm to approximately 2.0 mm. Patch 106 may be sufficiently sized to cover the target tissue with a margin for resection. Thus, patch 106 can come in many sizes to accomplish such a task. In some aspects, patch 106 may be approximately 50 mm by 50 mm (i.e., approximately 2 inches by 2 inches).

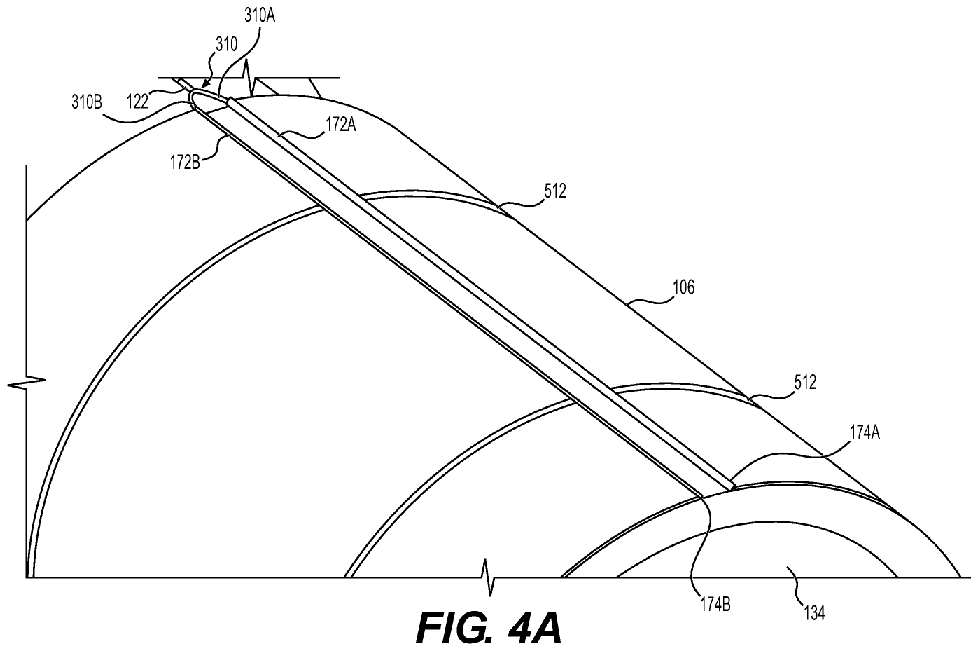

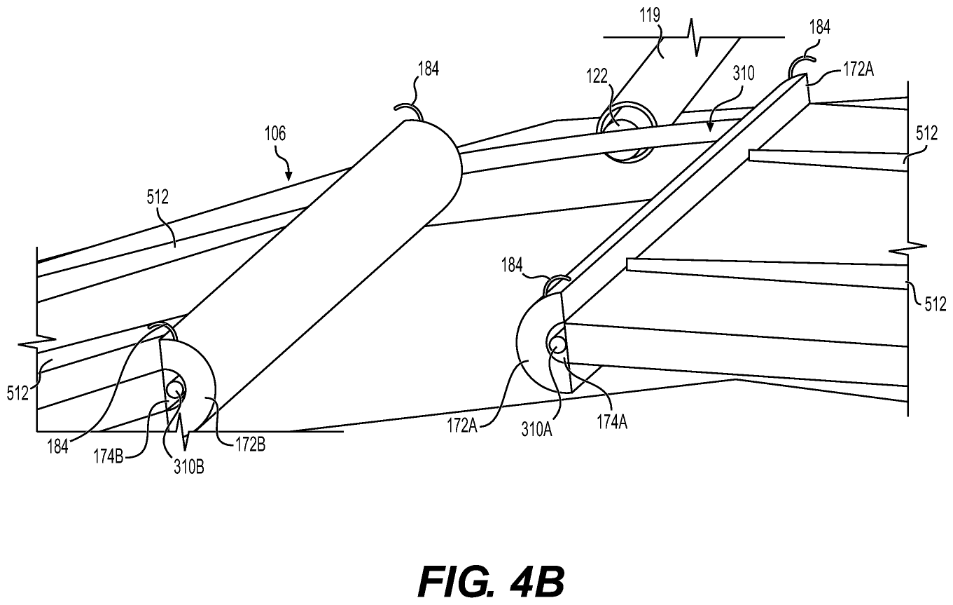

Patch 106 may be of any suitable color, including clear or at least partially transparent. Patch 106 may be formed of any suitable material, for example, nettings, meshes, cloths, gelatins, or polysaccharides (chitosan, cellulose, starch, alginates, etc.) that may be further modified with synthetic biocompatible materials (pHEMA, PGA, PLA, PCA, PEG, etc.). In some aspects, patch 106 may be formed of a bioadhesive material, for example, such as chitosan, modified chitosan, cellulose, pHEMA, PVA, PEG, or composites of one or more of these polymers. Additionally, for example, patch 106 may be comprised of polypropylene, polyester, Polytetrafluoroethylene (PTFE), expanded Polytetrafluoroethylene (ePTFE), and/or silicone. Patch 106 may be adhered to the target tissue using materials commonly known in the art, such as, for example, fibrin glue, hydrogel, and/or cyanoacrylate. Alternatively or additionally, patch 106 may be comprised of and/or dosed with agents to prevent the shedding of cells from the target tissue or to treat the target site. In some aspects, patch 106 may include a treatment agent, for example, an antibiotic and/or hemostatic agent. Moreover, after patch 106 is delivered to the treatment site, the user may spray, apply, or otherwise deliver one or more hemostatic agents (e.g., one or more hemostatic powders), for example, through a working channel (e.g., an internal lumen) of endoscope 102 or another medical device. Additional features of patch 106 are further discussed below when referring to FIGS. 4A and 4B, which illustrate an exemplary deployment mechanism of patch 106.

As shown in FIG. 1, medical device 104 includes end cap 108 and handle 110. One or more first sheath elements 118 and one or more second sheath element 119 may extend between handle 110 and end cap 108, for example, from one or more portions of handle 110 to one or more portions of end cap 108. Sheath element(s) 118, 119 may each be formed by a coil, a tube, a sheath, a hollow rod, etc.

Additionally, one or more control elements 120 and one or more actuation elements 122 may extend from one or more portions of handle 110 to one or more portions of end cap 108. Control element(s) 120 may each be formed by a coil, a tube, a sheath, a rod, etc. Actuation element(s) 122 may each be formed by a wire, a coil, a tube, a rod, etc. In these aspects, as shown in FIG. 3B, each control element 120 may be radially within a respective first sheath element 118, and each control element 120 may be movable within the respective first sheath element 118. Moreover, each actuation element 122 may be radially within a respective second sheath element 119, and each actuation element 122 may be movable within the respective second sheath element 119. Furthermore, as shown in FIGS. 4A and 4B, actuation element 122 may include a distal portion 310 that includes a first strand 310A and a second strand 310B. Each of first strand 310A and second strand 310B may be configured to engage a corresponding channel of patch 106, as discussed in further detail below.

As shown in FIGS. 1, 3A, and 3B, end cap 108 may include a stationary portion 126 and a scaffold or a movable portion 128. Stationary portion 126 may be coupled to distal portion 102B of endoscope 102, as shown in FIG. 3B. Stationary portion 126 may include a cross-sectional shape substantially corresponding to the cross-sectional shape of scope 102. In some aspects, stationary portion 126 may be cylindrical and/or ring-shaped with a lumen 130 (FIG. 1) extending longitudinally through stationary portion 126. Stationary portion 126 may be configured to be coupled to a radially-outward facing surface or outer surface of endoscope 102 (FIG. 3B). Stationary portion 126 may be coupled to distal portion 102B of endoscope 102, for example, via a friction fit, an adhesive, a press fit, a crimping, or any other appropriate coupling mechanism.

Moreover, as shown in FIG. 3B, stationary portion 126 may include one or more through-holes 132 extending longitudinally through portions of stationary portion 126 (i.e., radially outward of lumen 130). In some aspects, each of sheath element(s) 118, 119 may be coupled to a corresponding through-hole 132, for example, via a friction fit, an adhesive, a press fit, a crimping, or any other appropriate coupling mechanism. Additionally, control element(s) 120 (and actuation element 122) may extend longitudinally through a corresponding through-hole 132. In these aspects, control element(s) 120 (and actuation element 122) may move longitudinally (i.e., proximally and/or distally) relative to stationary portion 126 through corresponding through-holes 132, for example, to control a position of movable portion 128 relative to stationary portion 126 and/or to endoscope 102 (FIG. 3B).

Although not shown, stationary portion 126 (and/or movable portion 128) may include one or more radiopaque markers, protrusions, or indicators. The one or more radiopaque markers, protrusions, or indicators may be, for example, configured to facilitate visualization of stationary portion 126 (and/or movable portion 128), for example, via X-ray imaging, CT scan imaging, magnetic resonance imaging, or other external visualization techniques.

Movable portion 128 may be generally cylindrical, and may include a lumen 134 (FIG. 3B) extending longitudinally through movable portion 128. Moreover, movable portion 128 may include a proximal edge 136 to which control element(s) 120 may be coupled, fixed, or received at respective coupling points 142. The manner in which control element(s) 120 may be coupled to movable portion 128 is not particularly limited. As a result of such coupling, movement of control element(s) 120, for example, via one or more actions on handle 110, may control the position of movable portion 128 relative to stationary portion 126 and/or to distal portion 102B of endoscope 102. Moreover, movement of actuation element(s) 122, for example, via one or more different actions on handle 110, may control the position of patch 106. As shown in FIGS. 1 and 3B, patch 106 may be overlaid or otherwise positioned around one or more portions of movable portion 128.

As mentioned above, handle 110 may be coupled to end cap 108 via sheath element(s) 118, 119, control element(s) 120, and/or actuation element(s) 122. As shown in FIG. 1, sheath element(s) 118, 119 (as well as control element(s) 120, and actuation element(s) 122) may extend through a distal lumen 117 of handle 110 and be coupled to respective aspects of handle 110, as discussed in further detail below.

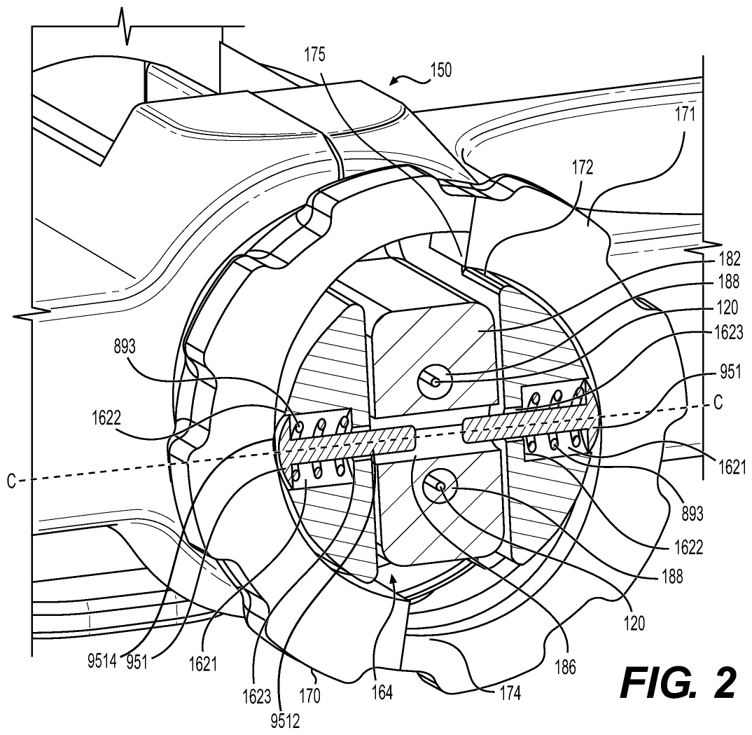

Handle 110 may include a handle body 160, a primary actuator 150, a second actuator 192, and a locking mechanism 170. Handle body 160 includes a longitudinal portion 162 that extends longitudinally. Longitudinal portion 162 includes a slot or track 164 extending along at least a portion of a length of longitudinal portion 162. Track 164 defines an internal passage that extends radially inwards from an outer surface of longitudinal portion 162, and said internal passage may be in communication with lumen 117. Track 164 may receive a riding portion 182 (shown in FIG. 2) of primary actuator 150, for example, that slidably translates along or within track 164, and track 164 may also receive secondary actuator 192, which may also slidably translate along or within track 164. Aspects of longitudinal portion 162 unencumbered by primary actuator 150 may be held within a hand of the user. Handle body 160 further includes an end portion 166, for example, at a proximal end of handle body 160. End portion 166 may define a finger hole 168 through which a user may insert or rest one or more fingers (e.g., the user's thumb), thereby securing the user's grip on handle body 160.

Primary actuator 150 may be a spool-like actuator that surrounds/sheaths a portion of longitudinal portion 162, and is configured to slidably translate along or within longitudinal portion 162, via track 164, in a proximal or distal direction (as indicated by the directional arrows P and D shown in FIG. 1.)

Primary actuator 150 may include additional features or aspects engaging track 164, thereby securing primary actuator 150 to longitudinal portion 162 and allowing for the slidable translation of primary actuator 150 within track 164. For example, as shown in FIG. 1, primary actuator 150 includes riding portion 182, which may be any suitable feature coupled to primary actuator 150 (as well as proximal portions of control element(s) 120), and also positioned within track 164. Thus, primary actuator 150 may translate along longitudinal portion 162, as riding portion 182 translates along or within track 164. Moreover, primary actuator 150 may include one or more finger holes, for example, a finger hole 150A and a second finger hole 150B. The user may insert at least one finger into each of finger holes 150A and 150B to rest the user's fingers or to apply force in a proximal or distal direction, thereby translating primary actuator 150 relative to longitudinal portion 162 (and riding portion 182 along track 164).

As noted above, riding portion 182 of primary actuator 150 may be coupled to control element(s) 120. For example, as shown in FIG. 2, riding portion 182 may be coupled to proximal portions or ends of control element(s) 120 such that the proximal portions or ends of control element(s) are held, for example, within a cavity or internal passages 188 of riding portion 182 (shown in FIG. 2). Riding portion 182 may be coupled to proximal portions or ends of control element(s) 120, for example, via a friction fit, an adhesive, a press fit, a crimping, or any other appropriate coupling mechanism. In this aspect, movement (i.e., proximal or distal movement) of riding portion 182 (and thus primary actuator 150) relative to handle body 160 may control the extension or retraction of control element(s) 120 relative to sheath element(s) 118, and thus control the extension or retraction movable portion 128 relative to stationary portion 126 (FIG. 3B). Riding portion 182 may further include a lumen 186 that extends across, or perpendicularly, to a longitudinal axis (i.e., the axis parallel to a length of track 164) of riding portion 182. Lumen 186 may engage features of an exemplary locking mechanism, as discussed in further detail below.

Although not shown, handle 110 may include one or more springs or biasing elements, for example, within the internal lumen of handle body 160, to bias the movement primary actuator 150. For example, the one or more springs or biasing elements may proximally bias the movement of primary actuator 150. In these aspects, the user may distally advance primary actuator 150 relative to handle body 160, but once the distal pressure from the user is removed, the one or more biasing elements may urge primary actuator 150 proximally. Additionally or alternatively, one or more portions of handle 110 may include a locking mechanism (e.g., locking mechanism 170), for example, to selectively and/or releasably secure a position of primary actuator 150. Because the position of primary actuator 150 dictates the position of end cap 108 (and patch 106 prior to deployment), primary actuator 150 may be secured to a distal position relative to handle body 160, via locking mechanism 170, to help stabilize the position of end cap 108 and patch 106 relative to the targeted site.

Locking mechanism 170 includes a knob-like actuator 171 that surrounds/sheaths a distal portion of longitudinal portion 162 and spring biased locking pins 951 (shown in FIG. 2). Actuator 171 may be distal to primary actuator 150, and may also serve as a stop, for example, limiting further distal translation of primary actuator 150 along track 164. Actuator 171 may be configured to rotate in a clockwise and/or counter-clockwise direction A (when viewed from a proximal end of handle 110), around longitudinal portion 162 (as indicated by the directional arrow shown in FIG. 1.)

As shown in FIG. 2, which illustrates a cross-section C-C (FIGS. 1 and 2) of medical device 104, a cross-section of actuator 171 may have an annular cam profile including an inner surface 174 and an indented portion 172. Inner surface 174 defines a gradual indentation that increasingly extends radially inwards (towards longitudinal portion 162) that terminates in indented portion 172. Indented portion 172 defines a stop surface 175, which may be configured to inhibit further rotation of actuator 171 in a direction, as stop surface 175 may abut against pin 951. As shown, actuator 171 may have a symmetrical cross-sectional profile including two indented portions 172 that are diametrically opposite of each other. Thus, the rotation, for example, clockwise rotation of actuator 171 (when viewed from a proximal end of handle 110) may result in the gradual indentation of inner surface 174 (and eventually indented portion 172) to increasingly abut against locking pins 951, thereby engaging locking pins 951 and applying pressure to a head 9514 of locking pin 951. In contrast, the rotation in another direction, for example, counter-clockwise rotation of actuator 171 (when viewed from a proximal end of handle 110) may result in increasing separation between indented portion 172 (and the gradual indentation of inner surface 174) and locking pins 951, thereby disengaging locking pins 951 and removing pressure applied to a head 9514 of locking pin 951. However, the direction of the rotation of actuator 171 to engage locking pins 951 or disengage from locking pins 951 is not particularly limited, and in other exemplary embodiments, counterclockwise rotation of actuator 171 may engage locking pins 951 and clockwise rotation of actuator 171 may disengage locking pins 951.

Locking pins 951 are not particularly limited, and may be any suitable pin, screw, or similarly shaped devices that generally include a longitudinal body 9512 and head 9514 protruding radially outwards relative to body 9512. Each of locking pins 951 may be housed within a cavity 1621 of handle body 160. Cavities 1621 may be on opposite sides of longitudinal portion 162 so that track 164 is positioned between cavities 1621 and bodies 9512 of pins 951 may extend towards track 164. Moreover, each of cavities 1621 includes an inner wall 1622 that encloses a portion of cavity 1621, and inner wall 1622 defines a through-hole 1623 that leads to track 164. Through-hole 1623 may be of a dimension configured to receive body 9512 of pin 951, and through-hole 1623 may be positioned so that it may be in coaxial alignment with lumen 186 of riding portion 182 when riding portion 182 is translated within track 164 to such a position. The relative position of riding portion 182 along track 164 so that lumen 186 is in coaxial alignment with through-holes 1623 is not particularly limited. In some instances, such coaxial alignment may be reached when primary actuator 150 abuts a proximal end of actuator 171, or is at a position that is adjacent to or near adjacent to actuator 171.

Each of locking pins 951 may be spring-biased, as shown in FIG. 2, as a spring 893 may surround at least a portion of body 9512. Spring 893 may be positioned between head 9514 and inner wall 1622. Thus, springs 893 may bias pins 951 away from track 164, thereby allowing for free translation of riding portion 182 (and primary actuator 150, as a result). However, pressure applied to head 9514 (via inner surface 174 and indented portion 172 of actuator 171) may compress spring 893, between head 9514 and inner wall 1622, and extend body 9512 through through-hole 1623 and towards track 164. Thus, when through-hole 1623 and lumen 186 are in coaxial alignment, pins 951, when compressed, inhibit further proximal or distal translation of riding portion 182, thereby securing primary actuator 150 into a locked position.

As noted above, secondary actuator 192 is coupled to longitudinal portion 162 and is configured to slidably translate along or within track 164, in a proximal or distal direction (as indicated by the directional arrows P and D shown in FIG. 1). Secondary actuator 192 is not particularly limited, and may be any suitable feature that is accessible to the user. Secondary actuator 192 may include any features (e.g., a protrusion, tab, etc.) which may help improve the manipulation of secondary actuator 192 along track 164. Additionally, secondary actuator 192 may be coupled to actuation element(s) 122, which may extend through lumen 117 and throughout a length of longitudinal portion 162 (e.g., via an internal passage). For example, an internal aspect (not shown) of secondary actuator 192 may be coupled to a proximal end of actuation element 122. Secondary actuator 192 may be coupled to the proximal end of actuation element 122, for example, via a friction fit, an adhesive, a press fit, a crimping, or any other appropriate coupling mechanism. In this aspect, proximal translation of secondary actuator 192, relative to handle body 160 (via track 164), may actuate the retraction of actuation element 122 towards secondary actuator 192, and thus control the deployment of patch 106.

Referring to FIGS. 4A and 4B, patch 106 and the deployable connection between patch 106 and actuator element 122 is further discussed. Patch 106 may be overlaid around at least a portion of an outer surface of movable portion 128 of end cap 108. Patch 106 includes a first edge 172A and a second edge 172B. Each of first edge 172A and second edge 172B may span at least a portion of the edges that define a length of patch 106. Thus, first edge 172A and second edge 172B may extend longitudinally along a portion of the length of end cap 108 when patch 106 is overlaid onto at least a portion of movable portion 128. In some aspects, first edge 172A includes a respective passage or a channel 174A, extending along at least a portion of first edge 172A. Likewise, in some aspects, second edge 172B includes a respective passage or a channel 174B, extending along at least a portion of second edge 172B. Each of channels 174A and 174B is not particularly limited, and may be of any suitable shape or dimension to receive and accommodate for first strand 310A and second strand 310B of distal portion 310 of actuation element 122. Channels 174A and 174B may receive, respectively, first strand 310A and second strand 310B, thereby helping to maintain or restrain patch 106 onto end cap 108. Thus, as locking mechanism 170 (FIG. 1) rotates clockwise and/or counter-clockwise, relative to handle body 160, actuation element 122 may translate proximally. Such proximal translation of actuation element 122 may retract first strand 310A and second strand 310B out from channels 174A and 174B, thereby releasing patch 106 from actuation element 122. As a result, patch 106 may be free to deploy from end cap 108 and be delivered onto the treatment site. As shown in FIG. 4B, each of first edge 172A and second edge 172B may further include one or more anchors 184. Anchors 184 is not particularly limited and may be any suitable feature, for example, hooks, adhesives, etc., configured to help anchor or otherwise secure patch 106 onto the treatment site. The positioning of anchors 184 along first edge 172A and second edge 172B is also not particularly limited, and in some instances, each of anchors 184 may be positioned on each of the ends of first edge 172A and second edge 172B.

Additionally, patch 106 may include one or more wires 512, each of wires 512 extending throughout a length of patch 106, for example, around a portion of a circumference of patch 106 wrapped around end cap 108. In some aspects, wires 512 may be embedded within or otherwise coupled to patch 106, and wires 512 may be biased towards a certain shape, for example, straight, circular, etc. Thus, the presence of wires 512 within or coupled to patch 106 may bias patch 106 into a corresponding shape. The general shape or contour of the treatment site may dictate the bias type of patch 106, so that patch 106 may be biased toward the appropriate shape or position when deployed onto the treatment site. The material of wires 512 is not particularly limited, and may be any suitable material that may flex and/or bias towards a certain shape, e.g., plastic, Nitinol, cobalt Nitinol, stainless steel, or any material(s) commonly known in the art.

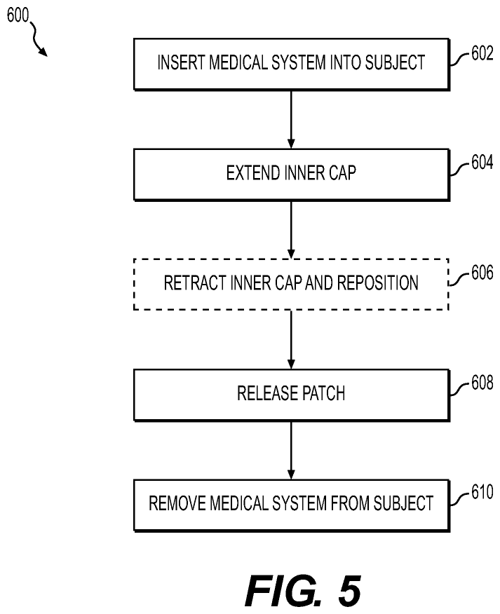

Referring to FIGS. 1, 2, 3A, 3B, 4A, 4B, and 5, an exemplary method 600 (FIG. 5) of operating medical system 100 is further discussed. During operation, a user may first couple medical device 104 to an insertion device, such as endoscope 102, for example, by coupling end cap 108 to distal portion 102B of endoscope 102. In some instances, handle 110 of medical device 104 may be coupled to a handle of endoscope 102 (e.g., a port of the handle). The user may then insert medical system 100 into the subject, as indicated in step 602 of method 600. The user may maneuver endoscope 102, using one or more visualization devices, illumination devices, etc. (not shown) at a distal tip of endoscope 102, to navigate distal portion 102B to a treatment site within a body of a subject.

As shown, once distal portion 102B of endoscope 102 and end cap 108 are positioned at the treatment site, the user may proximally retract sheath 112, for example, to expose movable portion 128 and patch 106 from sheath 112. Sheath 112 may extend proximally, for example, to handle 110, a proximal end of endoscope 102, etc. In some instances, a proximal aspect of sheath 112 may be accessible enough for the user to grab and pull, thereby manually retracting sheath 112. In some other instances, sheath 112 may include one or more pull wires, or other similar features, which may extend proximally for the user to pull and retract sheath 112. Alternatively or additionally, the user may extend end cap 108, as indicated in step 604. Specifically, movable portion 128 may be extended from stationary portion 126. As mentioned above, the user may manipulate (e.g., distally advance) primary actuator 150 (FIG. 1) relative to handle body 160 to extend movable portion 128. For example, manipulation of primary actuator 150 may distally advance control elements 120 to distally advance movable portion 128. Control elements 120 may move relative to sheath elements 118 (e.g., distally and/or proximally) and through stationary portion 126, for example, through a respective through-hole 132. Primary actuator 150 may be moved further distally and/or retracted proximally in order to extend or retract movable portion 128 and reposition movable portion 128 relative to the treatment site, as indicated in step 606. In some aspects, endoscope 102 may include one or more visualization devices and/or one or more illumination devices (not shown), which may help the user visualize the treatment site and/or the position of movable portion 128 relative to endoscope 102 and/or the treatment site. Once movable portion 128 is positioned relative to endoscope 102 and/or the treatment site, the user may lock or otherwise secure the position of primary actuator 150 via locking mechanism 170 and the rotation of actuator 171.

With movable portion 128 securely positioned (with the assistance of locking mechanism 170), the user may release patch 106, as indicated in step 608, by manipulating (e.g., translating) secondary actuator 192 (FIG. 1) in a proximal direction, relative to handle body 160, to retract actuation element 122 such that first strand 310A and second strand 310B of actuation element 122 are also retracted. As such, when actuation element 122 (and strands 310A and 310B) is proximally retracted out from channels 174A and 174B, patch 106 becomes unrestrained. Wires 512 may bias patch 106 toward a linear or circular position, for example, no longer wrapped around movable portion 128 of end cap 108. In these aspects, patch 106 may be deployed onto the treatment site into a position dictated by the bias of wires 512. Moreover, anchors 184 may latch or anchor onto the treatment site, or tissue surrounding the treatment site, for example, to help secure or otherwise position patch 106 on the treatment site.

Once patch 106 is deployed from end cap 108 and delivered to the treatment site, the user may inspect patch 106 and the treatment site, for example, using one or more visualization devices, illumination devices, etc. of endoscope 102. The user may also apply a hemostatic agent (e.g., a hemostatic powder or fluid) to the delivered patch 106. The user may also otherwise further attend to the treatment site, for example, via one or more auxiliary medical devices (e.g., a grasper) delivered through a working channel of endoscope 102. In some aspects, the one or more auxiliary medical devices may be used to position patch 106 on the treatment site. After deployment of patch 106 and any further treatment to the treatment site, the user may remove medical system 100 from the body of the subject, as indicated in step 610.

Various aspects of medical system 100, for example, medical device 104 with patch 106, end cap 108, and handle 110, may have a low cost and may be disposable (i.e., a single use device). Medical device 104 may be coupled to any type of scope to help deliver patch 106 to a treatment site (e.g., endoscopically), and coupling end cap 108 to the distal end of a scope may be quick and/or user-friendly. Patch 106 may be positioned on end cap 108 (e.g., radially around movable portion 128). Additionally, sheath 112 may help to protect patch 106 from fluids, tissues, materials, etc. during the delivery of endoscope 102 to the treatment site.

Additionally, medical device 104 may allow for patch 106 to be delivered to the treatment site in a minimally invasive procedure (e.g., endoscopically), without having to deliver patch 106 through a working channel (e.g., an internal lumen) of endoscope 102. In this aspect, patch 106 may be larger than patches passed through the working channel. Moreover, patch 106 and end cap 108 may not interfere with the delivery of one or more auxiliary medical device, delivery of fluid, application of suction, etc., which may be done through the working channel of endoscope 102. Furthermore, patch 106 may not interfere with the one or more illumination devices and/or visualization devices on an end face of endoscope 102. The extension/retraction of patch 106 may be done under direct visualization with the visualization devices of endoscope 102.

Moreover, patch 106 may be positioned relative to the treatment site using one or more auxiliary medical devices, and/or the user may inspect the position of patch 106 at the treatment site, for example, via one or more visualization device(s) on endoscope 102. The positioning and deployment of patch 106 (e.g., via primary actuator 150 and locking mechanism 170 of handle 110) may be straightforward and user-friendly, which may allow the user to be a surgical technician, while the physician performs one or more other tasks during the procedure.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a handle, wherein the handle includes a primary actuator and a secondary actuator;
an end cap configured to be coupled to a distal end of another medical device, wherein the end cap includes a stationary portion and a movable portion;
a patch, wherein the patch is coupled to the movable portion of the end cap;
one or more control elements coupling the primary actuator to the movable portion of the end cap; and
an actuation element, wherein the actuation element extends distally from the secondary actuator and the actuation element is configured to be coupled to the patch,
wherein movement of the primary actuator moves the movable portion of the end cap relative to the stationary portion of the end cap, and
wherein movement of the secondary actuator at least partially deploys the patch from the movable portion of the end cap.

2. The medical device of claim 1, wherein the handle includes a handle body, and wherein both the primary actuator and the secondary actuator are movable relative to the handle body to control the movement of the one or more control elements and the actuation element.

3. The medical device of claim 2, wherein the handle body includes a track and the primary actuator is movably coupled to track, and
wherein the primary actuator is configured to translate along the track in a first direction and an opposite second direction.

4. The medical device of claim 3, wherein a translation of the primary actuator in the first direction is configured to translate the movable portion of the end cap distally relative to the stationary portion, and wherein a translation of the primary actuator in the second direction is configured to translate the movable portion of the end cap proximally relative to the stationary portion.

5. The medical device of claim 1, wherein the patch comprises a first edge and an opposite second edge, wherein each of the first edge and the second edge includes a channel extending along at least a portion of the first edge or the second edge.

6. The medical device of claim 5, wherein the actuation element includes a proximal portion and a distal portion, and the distal portion is configured to be coupled to the patch.

7. The medical device of claim 6, wherein the distal portion includes a first strand and a second strand, and wherein the channel of the first edge of the patch is configured to receive the first strand and the channel of the second edge of the patch is configured to receive the second strand.

8. The medical device of claim 7, wherein the handle includes a track and the secondary actuator is movably coupled to track, and
wherein the secondary actuator is configured to translate along the track in a first direction, and wherein a translation of the secondary actuator in the first direction releases the actuation element from the patch and at least partially deploys the patch.

9. The medical device of claim 8, wherein the translation of the secondary actuator in the first direction pulls the actuation element in a proximal direction away from the patch, and retracts the first strand from the channel of the first edge of the patch and the second strand from the channel of the second edge of the patch.

10. The medical device of claim 5, wherein each of the first edge and the second edge of the patch includes one or more anchors, wherein each of the one or more anchors is configured to anchor the patch onto a treatment site.

11. The medical device of claim 10, wherein each of the one or more anchors comprise a hook.

12. The medical device of claim 1, wherein the patch comprises one or more wires, wherein each of the one or more wires extend throughout a length of the patch, and wherein the one or more wires bias the patch into a shape.

13. The medical device of claim 12, wherein the one or more wires are biased to a straight shape or to a circular shape.

14. The medical device of claim 1, wherein the stationary portion of the end cap includes one or more through-holes, and wherein the one or more control elements and the actuation element extend through the corresponding one of the one or more first through-holes of the stationary portion of the end cap.

15. The medical device of claim 1, further comprising a sheath configured to at least partially cover the patch and the end cap, and wherein the sheath is retractable and/or the movable portion is extendable to expose the patch and at least the movable portion of the end cap.

16. A medical device, comprising:
a handle, wherein the handle includes a primary actuator and a secondary actuator;
an end cap configured to be coupled to a distal end of another medical device, wherein the end cap includes a stationary portion and a movable portion;
a patch, wherein the patch is coupled to the movable portion of the end cap;
one or more control elements coupling the primary actuator to the movable portion of the end cap; and
an actuation element extending distally from the secondary actuator, wherein the actuation element includes a distal portion including a first strand and a second strand, and wherein the patch includes a first channel configured to receive the first strand and a second channel configured to receive the second strand,
wherein a movement of the secondary actuator is configured to retract the first strand from the first channel and the second strand from the second channel and at least partially deploy the patch from the movable portion of the end cap.

17. The medical device of claim 16, wherein movement of the primary actuator moves the movable portion of the end cap relative to the stationary portion of the end cap.

18. The medical device of claim 17, further comprising a locking mechanism, wherein the locking mechanism comprises a rotatable knob, and wherein a rotational movement of the rotatable knob secures the primary actuator in a locked position.

19. A medical system, comprising:
a scope, wherein the scope includes a distal portion;
a medical device, wherein the medical device includes:
a handle, wherein the handle includes a primary actuator and a secondary actuator;
an end cap coupled to the distal portion of the scope, wherein the end cap includes a stationary portion and a movable portion;
a patch coupled to the movable portion of the end cap, wherein the patch includes a first channel and a second channel;
one or more control elements coupling the primary actuator to the movable portion of the end cap; and
an actuation element extending distally from the secondary actuator, wherein the actuation element includes a first strand and a second strand, wherein the first strand is configured to be received by the first channel and the second strand is configured to be received by the second channel,
wherein movement of the primary actuator moves the movable portion of the end cap relative to the stationary portion of the end cap, and
wherein movement of the secondary actuator at least partially deploys the patch from the movable portion of the end cap.

20. The medical system of claim 19, wherein the handle includes a handle body,
wherein the handle body includes a track,
wherein both the primary actuator and the secondary actuator are movably coupled to the track,
wherein both the primary actuator and the secondary actuator are movable relative to the handle body, along the track, to control the movement of the one or more control elements and the actuation element, and
wherein the movement of the secondary actuator in a first direction releases the actuation element from the patch and at least partially deploys the patch.
